(19) United States
(12) Patent Application Publication
(10) Pub. No.: US 2026/0335648 A1
(43) Pub. Date: Oct. 29, 2026

(54) NEUROSTIMULATION SYSTEMS AND METHODS

(71) Applicant: Epic Neuro, Inc., Fountain Valley, CA (US)

(72) Inventors: James William PHILLIPS, Fountain Valley, CA (US); Robert M. ABRAMS, Fountain Valley, CA (US)

(21) Appl. No.: 19/103,712

(22) PCT Filed: Aug. 15, 2023
(86) PCT No.: PCT/US2023/072218

§ 371 Date: Feb. 13, 2025

Related U.S. Application Data
(60) Provisional application No. 63/371,496, filed on Aug. 15, 2022.

(51) Int. Cl. A61N 1/36; A61B 5/00; A61B 5/293; A61B 5/374; A61N 1/05; A61N 1/372; A61N 1/378

(52) U.S. Cl. CPC A61N 1/36135; A61B 5/0006; A61B 5/293; A61B 5/374; A61B 5/4064; A61B 5/4836; A61B 5/6847; A61N 1/0539; A61N 1/36125; A61N 1/36157; A61N 1/37211; A61N 1/378; A61B 2560/0214; A61B 2560/0468

(57) ABSTRACT

Systems and methods are described, which provides electrical stimulation to a person, while also facilitating EEG recording. The EEG recording can be analyzed to identify an intracerebral hemorrhage. In some examples, the EEG analysis can identify increased slow wave activity in a Delta frequency range of approximately 1-4 Hz and/or reduced Alpha activity in the Alpha frequency range of approximately 8-13 Hz. The systems and methods can include delivering electrical stimulation in response to the EEG analysis and/or notifying the person or a medical provider about the intracerebral hemorrhage.

### PRIORITY CLAIM

This patent application claims priority to U.S. provisional patent application No. 63/371,496, titled “NEUROSTIMULATION SYSTEMS AND METHODS”, filed Aug. 15, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Electric brain stimulation has been shown to be a potentially effective treatment for a number of brain disorders, including epilepsy, migraine, fibromyalgia, major depression, stroke rehabilitation, and Parkinson's disease. External stimulation tends to be unfocused, and direct cortical stimulation is often highly invasive, involving a craniotomy or drill holes in the skull in order to target a specific cortical location. It would be beneficial to find a brain stimulation solution that will provide targeted cortical stimulation without requiring a surgical procedure which penetrates the skull.

Electric brain stimulation may be accomplished by several means. Repetitive Transcranial Magnetic Stimulation (rTMS) is a noninvasive technique that uses a coil to deliver a series of high energy magnetic pulses to the brain, thereby inducing current to flow in the cortex underneath the coil. rTMS has been shown to be effective in the treatment of major depression, and other mental disorders. However, it is not easily directed to a particular location, and involves a large, expensive device to generate the high current pulse to the coil. rTMS is not portable and requires a treatment administrator to deliver therapy to the patient.

Transcranial Direct Current Stimulation (tDCS) uses electrodes on the outside of the head to deliver small amounts of current to the brain. tDCS was originally used for stroke recovery, and it has shown promise in the treatment of some mental disorders and for cognitive improvement. Electrodes are placed on skin surfaces on the outside of the subject's head near the region of interest for stimulation. The vast majority of current is shunted between the electrodes since the skull is a very effective electrical insulator. However, a portion of the current does result in intracerebral current flow, which may increase or decrease neuronal excitability and alter brain function. The exact method of action is unclear. tDCS current strength is limited due to the excitability of nerves in the scalp, which can cause discomfort to the patient if the current is set too high.

Vagus nerve stimulation involves electrically stimulating the vagus nerve in the neck of the patient. This can be done either using electrodes on the skin, which may involve painful sensation of the patient, or surgically implanting electrodes near the vagus nerve, generally with a power source implanted elsewhere in the body. This involves a significant surgical procedure and has shown efficacy in treatment of epilepsy and depression.

Deep brain stimulation (DBS) uses electrodes implanted and placed bilaterally into the basal ganglia, cerebellum, anterior principal nucleus, the centromedian nucleus, caudate nucleus, thalmic, or subthalmic region. Stimulation may also be delivered subcortically.

Stimulus trains are delivered for treatment of a number of disorders, including epilepsy, Parkinson's disease, and major depression. DBS is generally a very invasive procedure, requiring a long lead that penetrates the skull with multiple electrodes near the tip. The procedure is considered major surgery and is not generally used unless other methods have been exhausted.

Direct cortical stimulation (DCS) is similar to DBS, except that the lead lies on the surface of the cortex, either subdural or epidural. The electrodes are secured in place using sutures. This technique often involves removing a portion of the skull to gain access to the cortical surface, and possibly to make room for the power source. DCS has been shown to have efficacy in treatment of epilepsy and neuropathic pain. (Shanechi et al., 2013) introduced a Brain-Machine Interface that uses EEG to automatically titrate drugs during a medically induced coma. (Liu et al., 2006) automatically adjusted anesthetic during a surgical procedure using Bispectral index (BIS) calculated from the EEG. The company Aspect Medical, Inc. was created to develop a device for this application. Also, Drager Medical, Inc. developed the Zeus for closed-circuit anesthesia ventilation. (Doufas et al., 2003) used an automatic response test for optimizing propofol administration during conscious sedation. Phillips (U.S. Pat. Nos. 9,872,996, 10,780,286) uses a subcutaneous pulse generator and a conductive path through the skull at multiple locations to create a current-loop. The Phillips method and device still involves at least two drill-holes in the skull.

Around 40% of untreated aneurysms will eventually rupture. A rupture can be prevented with a technique called coiling, which closes off the blood flow into the aneurysm.

However, up to 5% of coiled aneurysms can still rupture. Aneurysmal subarachnoid hemorrhage (aSAH) has a 40-50% mortality rate, with most survivors dependent on others for daily living.

Delayed Cerebral Ischemia (DCI), the initial bleeding event following a ruptured brain aneurysm, is one of the most important causes of mortality and poor neurological outcome.

Neurological monitoring is essential for early DCI detection and intervention. By catching DCI early, pharmacologically-induced hypertension reverses the presenting deficit in 70% of patients. Clinical examination and intermittent transcranial Doppler ultrasonography and CT are the most commonly used to detect DCI, but they rely on patients coming to the clinic and scheduling precious resources, significantly delaying response.

### SUMMARY

A device for electrical stimulation of a subject's brain is provided, the device comprising: a case adapted to be implanted against a skull of the subject; a first electrode disposed on or in the case; a probe coupled to the case and configured to extend through a burr hole in the skull into the brain of the subject; a second electrode disposed on the probe and configured to deliver electrical stimulation to a target region of the brain and to sense electrical signals of the brain; and electronics disposed in the case and configured to generate electrical pulses, initiate an EEG recording of the subject's brain, and communicate with an external device; wherein the device is configured to record EEG signals of the brain in response to an EEG recording request from the external device.

In one aspect, the probe is flexible.

In some aspects, the device includes an insulating seal configured to fill a space between the burr hole and the probe. In one aspect, the insulating seal prevents fluid flow and electric current flow around the subcranial electrode.

In some aspects, the first electrode comprises a ring electrode. In some aspects, the ring electrode is integrated into the case.

In one aspect, the electronics are configured to generate electric current pulses between the first electrode and the second electrode.

In one aspect, the electric current pulses are configured to follow a path that proceeds from the second electrode through the target region, through a conductive path at a separate location in the skull from the burr hole, and underneath the scalp to the first electrode.

In some aspects, the electronics are further configured to analyze the EEG signals to identify an intracerebral hemorrhage in the target region.

In some aspects, analyzing the EEG signals further comprises identifying increased slow wave activity in a Delta frequency range of approximately 1-4 Hz.

In other aspects, analyzing the EEG signals further comprises identifying reduced Alpha activity in the Alpha frequency range of approximately 8-13 Hz.

In one aspect, the device is configured to wirelessly transmit the EEG signals to the external device.

In some aspects, the external device is further configured to analyze the EEG signals to identify an intracerebral hemorrhage in the target region.

In some aspects, analyzing the EEG signals further comprises identifying increased slow wave activity in a Delta frequency range of approximately 1-4 Hz.

In another aspect, analyzing the EEG signals further comprises identifying reduced Alpha activity in the Alpha frequency range of approximately 8-13 Hz.

In some aspects, the device is configured to wirelessly transmit the EEG signals to a cloud computing device.

In one aspect, the cloud computing device is further configured to analyze the EEG signals to identify an intracerebral hemorrhage in the target region.

In some aspects, analyzing the EEG signals further comprises identifying increased slow wave activity in a Delta frequency range of approximately 1-4 Hz.

In other aspects, analyzing the EEG signals further comprises identifying reduced Alpha activity in the Alpha frequency range of approximately 8-13 Hz.

A system is also provided comprising two or more of the devices recited in claim 1.

In some aspects, the two or more devices are configured to collectively record EEG signals of a subject's brain in response to an EEG recording request from an external device.

A system for electrical stimulation of a subject's brain is provided, comprising: a plurality of implantable neurostimulators configured to be implanted in a subject, each of the implantable neurostimulators including: a case adapted to be implanted against a skull of the subject; a first electrode disposed on or in the case; a probe coupled to the case and configured to extend through a burr hole in the skull into the brain of the subject; a second electrode disposed on the probe and configured to deliver electrical stimulation to a target region of the brain and to sense electrical signals of the brain; and electronics disposed in the case and configured to generate electrical pulses, initiate an EEG recording of the subject's brain, and communicate with an external device; wherein the system is configured to record EEG signals of the brain with the plurality of implantable neurostimulators in response to an EEG recording request from the external device.

In one aspect, the probes are flexible.

In some aspects, each stimulator of the system includes an insulating seal configured to fill a space between the burr hole and the probe. In one aspect, the insulating seal prevents fluid flow and electric current flow around the subcranial electrode.

In some aspects, the first electrode comprises a ring electrode. In some aspects, the ring electrode is integrated into the case.

In one aspect, the electronics are configured to generate electric current pulses between the first electrode and the second electrode.

In one aspect, the electric current pulses are configured to follow a path that proceeds from the second electrode through the target region, through a conductive path at a separate location in the skull from the burr hole, and underneath the scalp to the first electrode.

In some aspects, the electronics are further configured to analyze the EEG signals to identify an intracerebral hemorrhage in the target region.

In some aspects, analyzing the EEG signals further comprises identifying increased slow wave activity in a Delta frequency range of approximately 1-4 Hz.

In other aspects, analyzing the EEG signals further comprises identifying reduced Alpha activity in the Alpha frequency range of approximately 8-13 Hz.

In one aspect, the system is configured to wirelessly transmit the EEG signals to the external device.

In some aspects, the external device is further configured to analyze the EEG signals to identify an intracerebral hemorrhage in the target region.

In some aspects, analyzing the EEG signals further comprises identifying increased slow wave activity in a Delta frequency range of approximately 1-4 Hz.

In another aspect, analyzing the EEG signals further comprises identifying reduced Alpha activity in the Alpha frequency range of approximately 8-13 Hz.

In some aspects, the system is configured to wirelessly transmit the EEG signals to a cloud computing device.

In one aspect, the cloud computing device is further configured to analyze the EEG signals to identify an intracerebral hemorrhage in the target region.

In some aspects, analyzing the EEG signals further comprises identifying increased slow wave activity in a Delta frequency range of approximately 1-4 Hz.

In other aspects, analyzing the EEG signals further comprises identifying reduced Alpha activity in the Alpha frequency range of approximately 8-13 Hz.

A method of monitoring an aneurysm in a brain of a subject is provided, comprising: initiating an EEG recording in one or more implanted neurostimulator devices with an external device; analyzing the EEG recording to identify an intracerebral hemorrhage in the brain; and indicating to the subject or to a medical provider that the intracerebral hemorrhage has been identified.

In some aspects, prior to the initiating step, implanting one or more neurostimulator devices in the brain of the subject.

In some aspects, the method includes transmitting the EEG recording from the one or more implanted neurostimulator devices to a remote server.

In one aspect, the analyzing step is performed in the remote server.

In another aspect, the method includes generating a report on the analyzed EEG and transmitting the report to a medical provider of the subject.

In one aspect, the EEG recording is initiated with a smartphone, tablet, or PC.

In some examples, the analyzing step is performed locally on the one or more implanted neurostimulator devices.

In one example, analyzing the EEG recording further comprises identifying increased slow wave activity in a Delta frequency range of approximately 1-4 Hz.

In another example, analyzing the EEG recording further comprises identifying reduced Alpha activity in the Alpha frequency range of approximately 8-13 Hz.

In another example, the method includes providing electrical stimulation to the brain with the one or more implanted neurostimulator devices.

### BRIEF DESCRIPTION OF THE DRAWINGS

FIG. 1 shows a neurostimulator device.

FIGS. 2A-2B illustrate an EEG recording of a subject with an aneurysm.

FIG. 3 is a diagram showing one or more neurostimulators implanted near an aneurysm.

FIGS. 4A-4E illustrate a method of monitoring an aneurysm with one or more implanted neurostimulator(s).

FIGS. 5A-5B illustrate a series of EEG recordings of a subject with an aneurysm.

### DETAILED DESCRIPTION

While certain embodiments have been provided and described herein, it will be readily apparent to those skilled in the art that such embodiments are provided by way of example only. It should be understood that various alternatives to the embodiments described herein may be employed, and are part of the invention described herein.

The present disclosure provides a minimally invasive solution to on-demand cortical EEG recording. Quantitative EEG (qEEG) analysis provides evidence of an ischemic event, and can be detected using automated machine learning algorithm(s). Ischemia can cause an increase in slow-wave activity and a reduction in Alpha power in the region of the infarct.

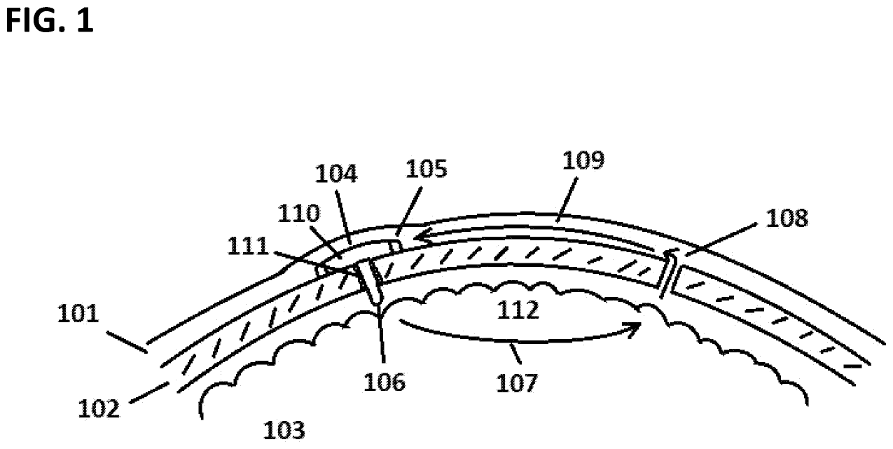

FIG. 1 shows one embodiment in which a neurostimulator device 104 is implanted beneath the scalp 101 of a subject and comprises a case 110, a probe-shaped subcranial electrode 106 which is inserted into a drill-hole in the skull 102, and a subcutaneous ring electrode 105 disposed around or integrated into the case 110. In one embodiment, the subcranial electrode 106 can comprise a screw adapted to be screwed into the subject's skull. The system can further comprise an electrically insulating seal 111 configured to fill the space between the device and the interior wall of the burr-hole, which prevents fluid flow and electric current flow around the subcranial electrode. The case 110 can be configured to rest on the surface of the skull and can comprise an electric current or voltage pulse generator to generate electric current pulses between the subcranial electrode and the subcutaneous electrode. Due to the high impedance of the skull 102, a majority of the electric current is forced to follow a path 107 which proceeds from the subcranial electrode through a target region 112 of the brain 103, through a conductive path 108 at a separate location in the skull from the burr hole, and back along path 109 underneath the scalp to the subcutaneous ring electrode 105.

In one embodiment, the device 104 can include electronics, such as a voltage or current pulse generator, configured to generate electric current waveforms between the subcranial electrode 106 and the subcutaneous ring electrode 105. The device can further include a power source such as a battery or a capacitor, or alternatively, can be powered externally with wireless power transfer (e.g., inductive coupling). The electronics can further include one or more processors, microcontrollers, or CPUs configured to control operation of the device and process and/or evaluate data sensed by the electrodes. In some embodiments, the electronics can further include memory configured to store recorded data and/or instructions related to the operation of the device and/or sensed parameters (e.g., EEG) of the patient. The electronics can be disposed or positioned within the case 110, for example. In some embodiments, the electronics are positioned external to the device and to the subject. In these embodiments, current pulses may be created external to the body, with percutaneous leads transmitting the current pulses to the subcutaneous/subcranial electrodes. The electronics can further include wireless communications electronics, to facilitate communication between the neurostimulator device and external devices. In some embodiments, the external devices can include smartphones, computers, tablets, or the like. In some embodiments, external devices can be configured to control operation of the neurostimulator device. For example, in one embodiment, a smartphone, tablet, or pc can be configured to turn on or off features of the neurostimulator device, such as initiating EEG recordings or stimulation therapy.

The device can be configured to record EEG and automatically determine the intrinsic frequency from the EEG recording and specify the pulse frequency, pulse amplitude, pulse shape, pulse width, or pulse duty cycle, and other parameters. The recorded EEG may also be transmitted wirelessly to an external module, such as a mobile device running a software application, where the software application determines the intrinsic frequency and specifies the pulse frequency, pulse amplitude, pulse shape, pulse width, or pulse duty cycle, and other parameters, and transmits the parameters to the device.

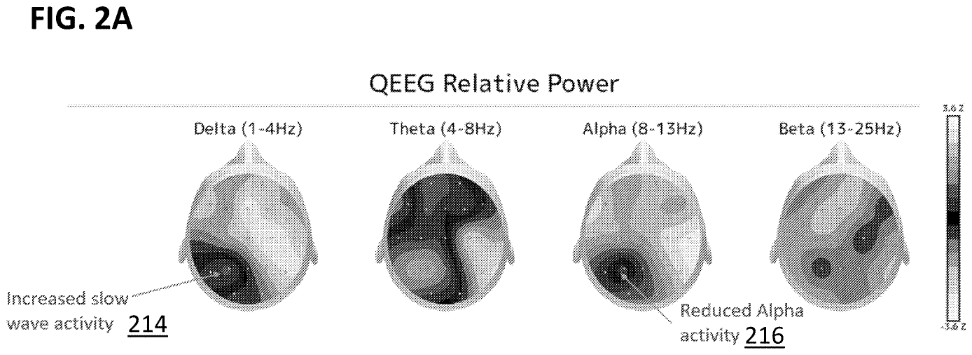

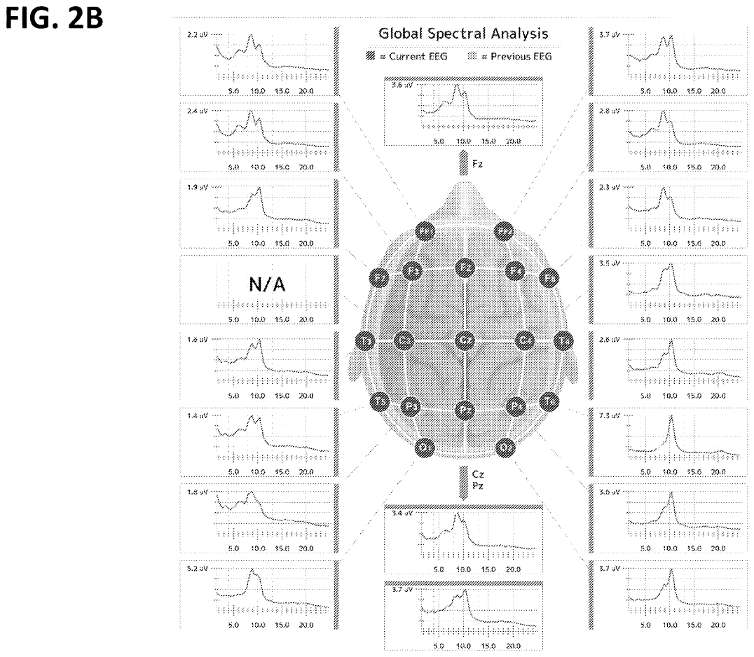

FIG. 2A shows EEG power distribution for a patient with an intracerebral hemorrhage localized in the left posterior region at a plurality of frequency ranges, as shown.

The frequency ranges can include a Delta frequency range (1-4 Hz), a Theta frequency range (4-8 Hz), an Alpha frequency range (8-13 Hz), and a Beta frequency range (13-25 Hz). As shown, an EEG of the brain indicates increased slow wave activity at the intracerebral hemorrhage location in the Delta frequency range (1-4 Hz) and reduced Alpha activity in the Alpha frequency range (8-13 Hz) as indicated by reference numbers 214 and 216, respectively. The devices of the present disclosure can be configured to identify regions in the brain with increased slow wave activity in the Delta frequency range and/or reduced Alpha activity in the Alpha frequency range to identify an intracerebral hemorrhage and/or other traumatic brain event.

FIG. 2B shows EEG recordings for the same patient taken at a plurality of locations within the brain, including locations F_{P1}, F_{P2}, F₃, F₄, F₇, F₈, F_{Z}, C_{Z}, C₃, C₄, T₃, T₄, T₅, T₆, P_{Z}, P₃, P₄, O₁, and O₂. Again, this detailed EEG plot shows alpha power drops and Delta/Theta power increases in the area of the intracerebral hemorrhage injury (e.g., in the left posterior region of the brain).

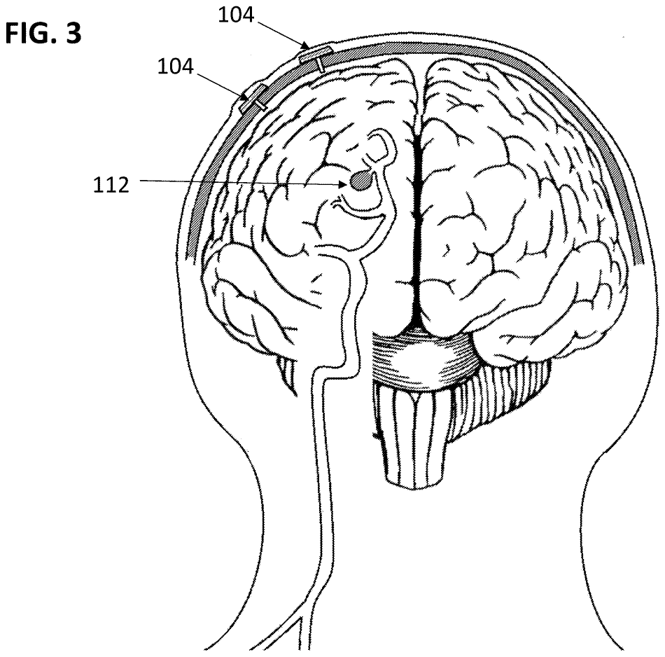

Referring to FIG. 3, one or more neurostimulator devices 104 (such as neurostimulator device 104 of FIG. 1) can be positioned above or adjacent to an aneurysm 112 in the patient's brain. By positioning the neurostimulator device(s) precisely near the aneurysm, the device(s) are able to record the highest quality EEG possible, right at the cortex of the brain. In some implementations, only a single neurostimulator device 104 is placed near the aneurysm. In other embodiments, a plurality of neurostimulator devices are placed near the aneurysm. The devices can each be individually configured to record EEG signals of the brain. In some embodiments, the devices can collectively or cooperatively collect and record EEG signals from the brain.

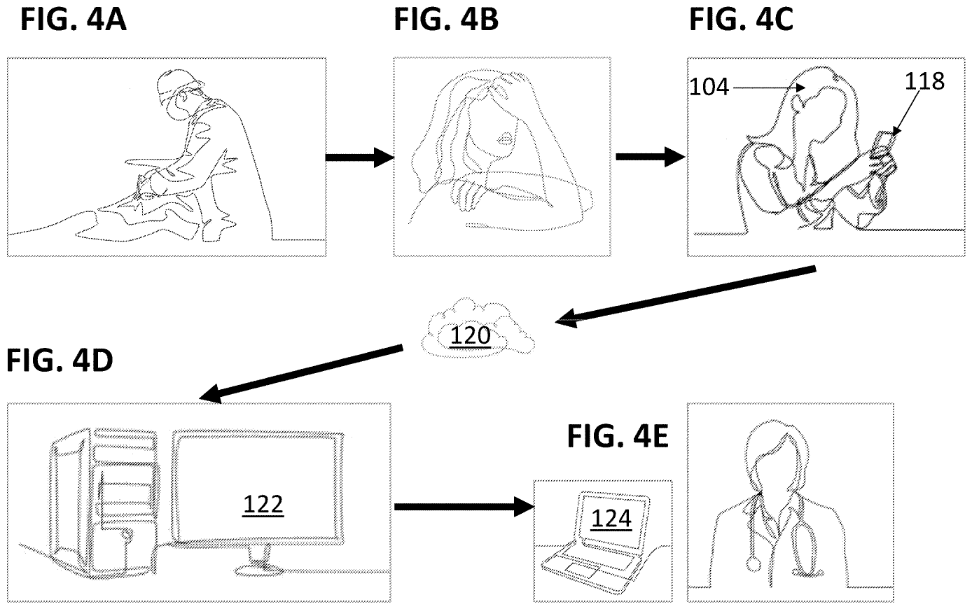

FIGS. 4A-4E illustrate a general sequence of events including the implantation and use of one or more neurostimulator devices for aneurysm monitoring. This procedure can be used for patients with a history or risk of aneurysm or other cerebral events. Referring to FIG. 4A, a surgeon or other medical provider can implant the neurostimulator device into the patient's brain at a location appropriate to an aneurysm. This procedure can be, for example, a simple 20 minute or less surgical procedure. In some embodiments, the implantation can be performed on an out-patient basis. In some embodiments, the neurostimulator can be implanted such that the probe-shaped subcranial electrode (from FIG. 1) is inserted into a drill-hole in the skull, and a subcutaneous ring electrode and/or case is positioned against the skull and under the scalp.

Referring to FIG. 4B, at some time after implantation, the patient may begin to experience symptoms of an aneurysm leak or rupture. For example, the patient may begin to feel symptoms associated with a ruptured aneurysm, including neck stiffness, drowsiness, confusion, dizziness, problems with balance, difficulty speaking, weakness or no feeling in an arm or a leg, etc.

Referring to FIG. 4C, the patient can initiate an EEG recording in the one or more implanted neurostimulator devices 104. In one example, the EEG recording can be initiated wirelessly via an external electronic device 118, such as a smartphone, tablet, or pc. In other embodiments, the patient may initiate the EEG recording by interacting directly with the neurostimulator device, such as by pushing a button on the device or on hardware or a lead that extends from the device to another location on the patient's body. Once initiated, the one or more implanted neurostimulators can be configured to record an EEG of the patient's brain. The EEG can be recorded for a pre-determined period of time. In some examples, the time of recording can be customized by a medical provider or by the user, such as with the external electronic device.

At FIG. 4D, the recorded EEG can be transmitted wirelessly, either from the implanted device itself or from the external device (e.g., the smartphone, tablet, or pc) to a remote or cloud based server 120 or to another computing system 122. This remote server can include one or more processors that are configured to automatically analyze the recorded EEG using one or more algorithms (including machine learning algorithms) to detect slow waves in the affected area and generate a report. The report can be, for example, an electronic report that includes details of the EEG recording(s) and or instructions or next steps for the patient or medical provider to perform. In some embodiments, the recorded EEG can be analyzed directly on the implanted stimulator, or alternatively, in the external device of the patient.

At FIG. 4E, the report can be transmitted to a medical clinic 124 or physician associated with the patient. For example, the transmission to the medical clinic 124 can include one or more computers, smartphones, or tablets of the medical clinic.

If the analysis of the recorded EEG indicates that a rupture or leak has occurred, the clinic, medical provider, and/or patient can be alerted. The clinic and/or medical provider can be instructed to contact the patient for immediate evaluation and attention to the ruptured or leaking aneurysm.

In some embodiments, the implanted neurostimulator device(s) can be adapted and configured to immediately provide stimulation therapy to the region of the ruptured or leaking aneurysm in response to the EEG analysis or electronic report. Not only are the implanted device(s) ideally positioned to record EEGs relating to the aneurysm, but they are also optimally positioned within the brain to potentially treat the aneurysm with stimulation therapy. As such, in some embodiments, stimulation can be manually or automatically initiated in response to the EEG recording and analysis identifying a ruptured or leaking aneurysm. In some embodiments, the patient can initiate therapy, such as through the external device (e.g., smartphone, tablet, pc). In other embodiments, the medical provider can remotely initiate therapy after reviewing the report of the EEG. Alternatively, the system can be configured to automatically initiate therapy in response to identifying a rupture or leaking aneurysm.

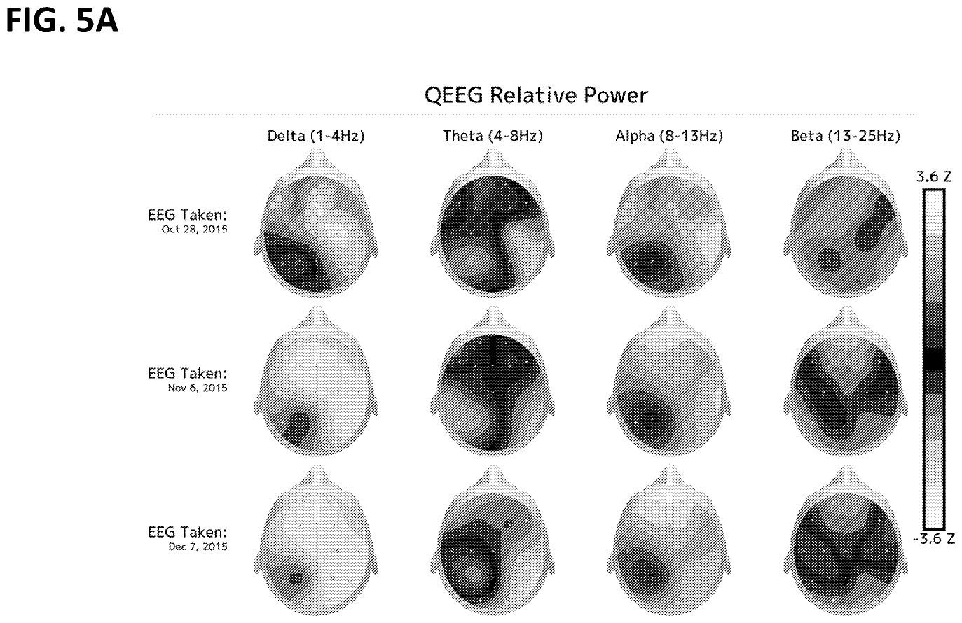

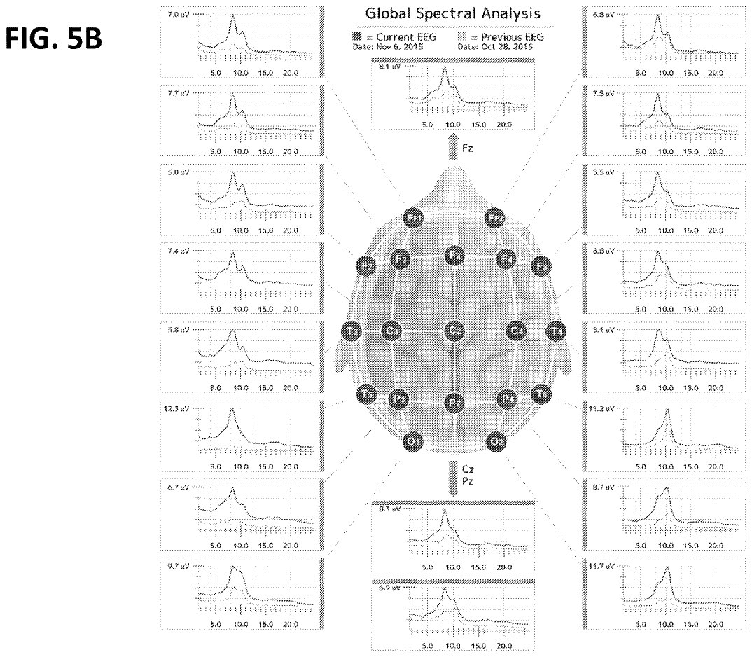

FIG. 5A shows a series of EEG recorded with implanted neurostimulators over a period of time in a patient with an intracerebral hemorrhage localized in the left posterior region (as in FIG. 2A). In this example, the three EEG recordings occur over the course of about 6 weeks. Stimulation therapy was provided to the patient over this period with the implanted neurostimulators described herein, and follow-up and the EEG recordings revealed improved speech, motivation, sleep, and improvement in sensation. FIG. 5B is a detailed EEG plot showing improved Alpha activity across the entire region. Slow wave activity has decreased significantly in comparison to normal rhythmic Alpha waves.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present.

Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others.

Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure.

Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A device for electrical stimulation of a subject's brain, the device comprising:
a case adapted to be implanted against a skull of the subject;
a first electrode disposed on or in the case;
a probe coupled to the case and configured to extend through a burr hole in the skull into the brain of the subject;
a second electrode disposed on the probe and configured to deliver electrical stimulation to a target region of the brain and to sense electrical signals of the brain; and
electronics disposed in the case and configured to generate electrical pulses, initiate an EEG recording of the subject's brain, and communicate with an external device;
wherein the device is configured to record EEG signals of the brain in response to an EEG recording request from the external device.

2.-4. (canceled)

5. The device of claim 1, wherein the first electrode comprises a ring electrode.

6. (canceled)

7. The device of claim 1, wherein the electronics are configured to generate electric current pulses between the first electrode and the second electrode.

8. The device of claim 1, wherein the electric current pulses are configured to follow a path that proceeds from the second electrode through the target region, through a conductive path at a separate location in the skull from the burr hole, and underneath the scalp to the first electrode.

9. (canceled)

10. The device of claim 1, further comprising a power source disposed in the case.

11. The device of claim 1, wherein the electronics are further configured to analyze the EEG signals to identify an intracerebral hemorrhage in the target region.

12. The device of claim 11, wherein analyzing the EEG signals further comprises identifying increased slow wave activity in a Delta frequency range of approximately 1-4 Hz.

13. The device of claim 11, wherein analyzing the EEG signals further comprises identifying reduced Alpha activity in the Alpha frequency range of approximately 8-13 Hz.

14. The device of claim 1, wherein the device is configured to wirelessly transmit the EEG signals to the external device.

15. The device of claim 14, wherein the external device is further configured to analyze the EEG signals to identify an intracerebral hemorrhage in the target region.

16. The device of claim 15, wherein analyzing the EEG signals further comprises identifying increased slow wave activity in a Delta frequency range of approximately 1-4 Hz.

17. The device of claim 15, wherein analyzing the EEG signals further comprises identifying reduced Alpha activity in the Alpha frequency range of approximately 8-13 Hz.

18.-23. (canceled)

24. A system for electrical stimulation of a subject's brain, comprising:
a plurality of implantable neurostimulators configured to be implanted in a subject, each of the implantable neurostimulators including:
a case adapted to be implanted against a skull of the subject;
a first electrode disposed on or in the case;
a probe coupled to the case and configured to extend through a burr hole in the skull into the brain of the subject;
a second electrode disposed on the probe and configured to deliver electrical stimulation to a target region of the brain and to sense electrical signals of the brain; and
electronics disposed in the case and configured to generate electrical pulses, initiate an EEG recording of the subject's brain, and communicate with an external device;
wherein the system is configured to record EEG signals of the brain with the plurality of implantable neurostimulators in response to an EEG recording request from the external device.

25.-27. (canceled)

28. The system of claim 24, wherein each first electrode comprises a ring electrode.

29. Th system of claim 24, wherein each ring electrode is integrated into the case.

30. The system of claim 24, wherein the electronics are configured to generate electric current pulses between the first electrode and the second electrode.

31. The system of claim 24, wherein the electric current pulses of each implanted neurostimulator are configured to follow a path that proceeds from the second electrode through the target region, through a conductive path at a separate location in the skull from the burr hole, and underneath the scalp to the first electrode.

32. The system of claim 24, wherein the electronics of each implanted neurostimulator are further configured to analyze the EEG signals to identify an intracerebral hemorrhage in the target region.

33.-42. (canceled)

43. A method of monitoring an aneurysm in a brain of a subject, comprising:
initiating an EEG recording in one or more implanted neurostimulator devices with an external device;
analyzing the EEG recording to identify an intracerebral hemorrhage in the brain; and
indicating to the subject or to a medical provider that the intracerebral hemorrhage has been identified.

44.-50. (canceled)

51. The method of claim 43, wherein analyzing the EEG recording further comprises identifying reduced Alpha activity in the Alpha frequency range of approximately 8-13Hz.

52. The method of claim 43, further comprising providing electrical stimulation to the brain with the one or more implanted neurostimulator devices.
